(19) United States
(12) Patent Application Publication
(10) Pub. No.: US 2021/0106705 A1
(43) Pub. Date: Apr. 15, 2021

(54) Assembly and Method for Decontaminating Objects

(71) Applicant: Relyon Plasma GmbH, Regensburg (DE)

(72) Inventor: Stefan Nettesheim, Regensburg (DE)

(21) Appl. No.: 17/131,070

(22) Filed: Dec. 22, 2020

Related U.S. Application Data
(63) Continuation of application No. PCT/IB2019/054898, filed on Jun. 12, 2019.

(30) Foreign Application Priority Data
Jun. 26, 2018 (DE) 10 2018 115 300.7

(51) Int. Cl. A61L 2/14; A61L 2/24

(52) U.S. Cl. CPC A61L 2/14; A61L 2/24; A61L 2202/14; A61L 2202/11; A61L 2202/122

(57) ABSTRACT

The invention relates to an assembly and a method for completely sterilizing at least one object. For this purpose, a chamber is provided in which the object is sterilized. A plasma system for producing reactive species is associated with the chamber. A conveying means is arranged in such a way that a circulation mass flow leads through the plasma system and over the at least one object through the chamber and back to the conveying means. An outlet (8) is fluidically associated with the chamber such that an effective mass flow (8W) can be led out of the chamber.

### CROSS-REFERENCE TO RELEGATED APPLICATIONS

The present application is filed under 35 U.S.C. §§ 111(a) and 365(c) as a continuation of International Patent Application No. PCT/IB2019/054898, filed on Jun. 12, 2019, which application claims priority from German Patent Application No. DE 10 2018 115 300.7, filed on Jun. 26, 2018, which applications are incorporated herein by reference in their entireties.

### FIELD OF THE INVENTION

The present invention relates to an assembly for decontaminating at least one object. The assembly comprises a chamber in which the at least one object to be treated is placed. A plasma system is fluidically connected to the chamber for the object to be treated.

The invention also relates to a method for decontaminating at least one object.

### BACKGROUND OF THE INVENTION

In industry, plasma processes are currently used in, amongst others, medical technology, material production and lighting technology. In principle, the use of plasma allows a reduction in microbial contaminants at low temperatures, the effect primarily being achieved on the surfaces. First series of tests on a laboratory scale for plasma application in the food sector mainly investigate possibilities for inactivating undesirable microorganisms in heat-sensitive foods, since conventional thermal decontamination methods cannot be used or can only be used to a limited extent for products such as fresh fruit and vegetables, meat or eggs. The application of plasma is also a potential alternative to other chemical methods (e.g., use of chlorine) or physical methods (e.g., high pressure, high voltage pulses, ionizing radiation). The advantages of the plasma method comprise, amongst others, a high effectiveness at low temperatures (generally <70° C.), targeted and consumption-based provision, low impact on the inner product matrix, water-free, solvent-free and residue-free as well as resource-efficient use. Other methods, such as high pressure and/or ionizing radiation, are complex or costly. Decontamination with UV light is often not effective and is limited by shadow effects.

International Patent Application WO 1995/009256 A1 relates to the treatment, in particular the cleaning, of surfaces, in particular film and metal surfaces. Cleaning is achieved in that a dielectrically impeded discharge acts on the surface of the foil strip. By the action of high-energy charged particles and by the action of photons from the UV spectral range, e. g. the oil or grease, film is removed so far after a short time that further processing is possible.

European Patent EP 1 337 281 B1 describes an enhancement of the cleaning effect of plasma. The enhancement of the indirect effect on the surface to be cleaned or sterilized is achieved by adding additive gas components, such as oxygen, water vapor or noble gases.

German Patent Application DE 100 36 550 A1 relates to a sterilization method in which the surface to be treated is exposed to a gas discharge. The sterilization is carried out in a gas atmosphere containing hydrogen and oxygen at a certain pressure. An optimum effect is achieved in particular by humidified air (N₂+O₂+H₂O).

U.S. Patent Application US 2015/0038584 A1 discloses a device for plasma treatment of surfaces of objects. A spatial and temporal separation of the plasma process and the exposure of the object to be cleaned and/or sterilized is described.

U.S. Patent Application US 2016/0220714 A1 discloses a disinfection device for plasma disinfection of surfaces with a plasma generator. In order to generate a disinfecting plasma gas stream, the plasma gas stream is in a communicating connection with the plasma generator. An at least partially closed disinfection region is provided, which is designed to accommodate the surface to be disinfected. The disinfection device has an aerosol generator for generating an aerosol stream containing aqueous particles. The aerosol generator is in communicating connection with the plasma generator in order to guide the plasma gas stream mixed with the aerosol stream in the disinfection region onto the surface to be disinfected.

Depending on the process for the sterilization, further additives such as peracetic acid or hydrogen peroxide, can be added.

U. S. Patent Application US 2003/0133832 A1 discloses the use of free hydroxyl radicals for sterilization or decontamination. The hydroxyl radicals have a particularly high oxidation potential. The hydroxyl radicals are formed by the photolytic reaction of ozone with water under UV light.

German Patent Application DE 10 2008 037 898 A1 relates to a method and a device for disinfecting or sterilizing packaging material and/or containers and/or filter material, the material or the container being treated with a gas generated in a plasma reactor.

German Patent Application DE 10 2015 119 369 A1 relates to a device as well as a system and a method for treating an object, in particular one or more free-form bodies, with plasma. The device is used to treat an object with plasma and comprises a casing device with which a substantially gas-tight receiving space can be formed or can be configured, in which an object to be treated can be accommodated. Furthermore, the device comprises a first electrode and a second electrode, the two electrodes being arranged in relation to the casing device in such a manner that when an electrical potential difference is applied to the electrodes, a plasma can be generated in the receiving space of the casing device.

U.S. Patent Application US 2017/112157 A1 discloses a method for treating a surface with a reactive gas. The reactive gas is produced from cold plasma at high voltage from a working gas (HVCP).

German Patent Application DE 10 2014 213 799 A1 discloses a household refrigeration device with a food treatment unit and a method for operating such a household refrigeration device. The household refrigerator is provided with an interior space for accommodating food, which is delimited by the walls of an inner container. Furthermore, a food treatment unit is provided, the food treatment unit being arranged in the household refrigerator to act on a surface of the food brought into the storage area and configured so that the acting is a decontamination of pesticides and/or heavy metals in the food.

German Patent Application DE 10 2005 061 247 A1 discloses a method and a device for sterilizing food. The food is exposed to at least one atmospheric plasma jet. The energy contained in the plasma jet disinfects the surface of the food.

U.S. Patent Application US 2004/002673 A1 discloses a sterilization method that is not carried out in a vacuum. First, an oxidizing agent is introduced in a gaseous state into a sterilization chamber or an area to be sterilized. A gas plasma is also introduced into the sterilization chamber or the area to be sterilized.

U.S. Pat. No. 6,228,330 B1 discloses a decontamination/sterilization chamber for atmospheric pressure plasma. The decontamination/sterilization chamber is used to decontaminate sensitive equipment and materials, such as electronics, optics and national treasures, which have been contaminated with chemical and/or biological warfare agents, such as anthrax or blistering agent. The device may also be used for sterilization in the medical and food industries. Items to be decontaminated or sterilized are placed in the chamber. Reactive gases containing atomic and metastable oxygen species are generated by an atmospheric-pressure plasma discharge in an He/O₂ mixture and directed into the area of these objects, resulting in a chemical reaction between the reactive species and organic substances. The plasma gases are recirculated through a closed-loop system to minimize the loss of helium and the possibility of escape of aerosolized harmful sub stances.

U. S. Patent Application US 2014/0322096 A1 discloses a sanitization station with a fluid source and one or more plasma generators for generating non-thermal plasma. One or more nozzles spray a mist or stream of fluid through plasma generated by the one or more plasma generators to activate the fluid. The liquid is then used to sterilize an object.

International Patent Application WO 2008/126068 A1 discloses a plasma system with a plug-in cartridge, a pair of identical opposing electrode blocks, and an inlet conduit for process gas. The process gas, such as air, is drawn into the conduit in a flow and flows in a flow direction along the path on which it is ionized. The ionized air emerges in a flow into the treatment line, where it hits the sample to be treated.

U.S. Patent Application US 2013/0071286 A1 discloses a sterilization device by means of cold plasma for the sterilization of objects, such as medical instruments. The gas is fed to a plasma chamber, where it is excited by one or more electrodes coupled to a pulse source to thereby generate a cold plasma within the plasma chamber. A dielectric barrier is disposed between the gas chamber and the electrodes to form a dielectric barrier discharge device. Inside the plasma chamber, one or more conductive posts connected to the floor hold the object to be sterilized. The cold plasma leaves the plasma chamber, where it is returned for further use as a plasma source in subsequent cycles.

All non-thermal processes (chemical, plasma-chemical, physical or optical) are more effective the higher the concentration of the active species and the higher the exposure time of the contaminated surface is. Threshold values are often observed for microbiological organisms, below which the effect disappears even after prolonged exposure. A complete decontamination cannot be achieved in this way.

In the case of direct treatment with chemical gas mixtures or liquids, such as ethylene oxide or H₂O₂, the concentration can be freely adapted to the process within a wide range. However, concentrations that are hazardous to health must then be handled safely.

In the case of UV or discharge processes, the concentration of the species produced is heavily dependent on the power and power density, which cannot be increased at will.

### SUMMARY OF THE INVENTION

An object of the invention is to provide an assembly for decontaminating or sterilizing objects, by means of which the decontamination or sterilization can be carried out effectively and in a resource-saving manner in the shortest possible time.

This object is achieved by an assembly for decontaminating or sterilizing at least one object, which assembly comprises a chamber in which the at least one object is placed; a plasma system which is fluidically connected with the chamber; a mixing chamber, which is arranged upstream of the plasma system and connected via a pipe to an inlet of a discharge chamber of the plasma system, such that an input mass flow can be supplied to the discharge chamber of the plasma system; and a conveying means which is arranged in a second supply pipe from the chamber to the mixing chamber in order to guide a circulation mass flow from the chamber back to the mixing chamber.

A further object of the invention is to provide an automatic and controlled method for decontaminating or sterilizing objects, wherein the decontamination or sterilization can be carried out effectively and in a resource-saving manner in the shortest possible time.

This object is achieved by a method for decontaminating or sterilizing objects which comprises the following steps: charging a discharge chamber of a plasma system with a gas mixture from at least one mixing chamber via a pipe; igniting a discharge with the gas mixture in the discharge chamber of the plasma system; feeding an acting mass flow from the discharge chamber to a chamber via pipes; guiding a recirculation mass flow from the chamber to the mixing chamber with a conveying means in a second supply pipe, and feeding again the recirculation mass flow to the chamber via the discharge chamber of the plasma system, so that an acting mass flow leaving the discharge chamber has an increased concentration of reactive compounds or substances; and controlling at least one voltage source of the discharge chamber of the plasma system, the mixing chamber and the conveying means by means of a control and measuring unit for process control, and the control and measuring unit for process control collecting data at least from the discharge chamber of the plasma system, the mixing chamber and the conveying means, which data are used to control the process management.

In embodiment of the assembly for decontaminating or sterilizing at least one object, a chamber is provided which is fluidically connected to a plasma system. The object to be treated is placed in the chamber. A mixing chamber is arranged upstream of the plasma system. The mixing chamber is connected to an inlet of a discharge chamber of the plasma system via a pipe, so that an input mass flow can be fed to the discharge chamber of the plasma system. A conveying means is arranged in a second supply pipe from the chamber to the mixing chamber. A circulation mass flow from the chamber can be fed back to the mixing chamber by means of the conveying means. The conveying means is preferably designed as a pump.

The advantage of using the circulation mass flow is that reactive compounds or substances are repeatedly conveyed through the discharge chamber of the plasma system, so that their concentration increases with each passage up to a certain equilibrium. As a result, the concentration required for effective decontamination or sterilization can be obtained with a relatively short or not too long discharge chamber of the plasma system. The discharge chamber of the plasma system may be designed as a flow-through reactor that is charged with a gas mixture. A discharge is ignited in this flow-through reactor. Depending on the intensity of the gas discharge, the gas composition and other process parameters, such as throughput rate (flow-through rate, flow rate), temperature or pressure, a composition of products (reactive compounds or substances) with different lifespans and reactivity. In chemical process engineering, such a flow-through reactor is also known as a continuous stirred-tank reactor (CSTR).

A conveying means is designed, for example, as a pump or fan, and may be arranged in such a way that a circulation mass flow is guided through the discharge chamber of the plasma system and over at least one object in the chamber (and back). An outlet may be fluidically associated with the chamber, so that an effective mass flow can be discharged from the chamber. In the event that the conveying means is a pump, this can be designed as a membrane pump.

As a result of the recirculation initiated by means of the conveying means, the yield of the discharge chamber of the plasma system or the assembly according to the invention can be increased considerably. The composition of the products that act on the object can be adjusted within a wide range. A dielectric barrier discharge may be used as the discharge type in the plasma system or in the discharge chamber. This can keep the required power low. According to an embodiment, at least one piezoelectric transformer is provided in the discharge chamber of the plasma system. The piezoelectric transformer is connected to the voltage source of the plasma system for generating plasma and the reactive species.

According to an embodiment of the invention, the discharge chamber of the plasma system and the chamber with the object are spatially separated from one another.

With the same basic concept, the various embodiments of the assembly differ only in the degree of integration of the various components (chamber, mixing chamber, recirculation turnout, conveying means, heat sources and/or heat sinks, dosing units, etc.) and the details of the process control (discharge capacity, temperatures and mass flows).

In the simplest embodiment, the assembly consists of a chamber in which the discharge burns. A recirculating flow is maintained within the chamber. An equilibrium of the concentrations is established within a certain time if the boundary conditions are fixed. If a liquid or aqueous phase and a gaseous phase (air) coexist in the chamber and if the pH value in the liquid or aqueous phase decreases into the acidic range, the concentration of hydrogen peroxide increases up to a given equilibrium value. By supplying and renewing air and water in a suitable ratio, the closed reactor becomes a flow-through reactor.

In the simplest case, the pump may even be omitted. The conveying means is thus formed solely by the power input of the discharge, so that a convection flow is driven in the chamber.

In an embodiment, a recirculation turnout is provided between the plasma system and the chamber. The recirculation turnout is fluidically connected to an outlet of the plasma system or the discharge chamber via a pipe. A first outlet of the recirculation turnout is fluidically connected to the circulation mass flow and a second outlet of the recirculation turnout is fluidically connected to the chamber via a pipe.

An inlet of the discharge chamber is fluidically assigned to the plasma system, so that an input mass flow can be fed to the plasma system. With the plasma system, the ratio of freshly supplied media (input mass flow) and returned products (recirculation ratio) can be set. A control and measuring unit is provided for this purpose. The recirculation ratio is between 1:10 and 10:1, but typically 1:3. To set the recirculation ratio, according to an embodiment, a recirculation turnout is provided between the plasma system and the chamber.

A heat source and/or heat sink may be associated with the plasma system for temperature monitoring and/or temperature setting. A heat source and/or heat sink may also be associated with the chamber for temperature monitoring and/or temperature setting. In addition, a condensate separator may be associated with the chamber.

To feed compounds or substances into the discharge chamber of the plasma system, a mixing chamber may be fluidically connected to the plasma system via the inlet. The mixing chamber may be designed as an evaporator or humidifier. A heat source and/or heat sink may also be associated with the mixing chamber. The mixing chamber itself is connected to at least one dosing unit via a first supply pipe and to the conveying means via a second supply pipe. The compounds or substances freshly supplied from the dosing units may be process gas, e.g. air or humidified air. An additional process gas, e.g. water vapor or oxygen, hydrogen peroxide or an organic vapor, may be used.

For process control, the assembly may be assigned a control and measuring unit which is communicatively connected to elements of the assembly. Elements of the assembly are, for example, the voltage source, the recirculation turnout, the chamber, the plasma system, the conveying means, the mixing chamber, the heat sources and/or heat sinks or the at least one dosing unit.

The heat sources and/or heat sinks used in the device for decontamination or sterilization may be implemented in various ways. For example, the plasma process or the discharge chamber itself may be a heat source. The heat gained in this way can be used to evaporate a liquid component. The evaporation process is therefore a heat sink. This relationship has the advantage that the discharge chamber or the plasma reactor is cooled by the evaporation. Likewise, no additional heating power needs to be used to evaporate a liquid medium (e.g., water). In an analogous manner, this principle can also be applied to the heat source and/or heat sink of the mixing chamber and/or the heat source and/or heat sink of the recirculation turnout and/or heat source and/or heat sink of the chamber itself.

The method according to the invention for decontaminating objects comprises the following steps:

charging a discharge chamber of a plasma system with a gas mixture from at least one mixing chamber via a pipe;

igniting a discharge with the gas mixture in the discharge chamber of the plasma system;

feeding an acting mass flow from the discharge chamber to a chamber via pipes;

guiding a recirculation mass flow from the chamber to the mixing chamber with a conveying means in a second supply pipe, and feeding again the recirculation mass flow to the chamber via the discharge chamber of the plasma system, so that an acting mass flow leaving the discharge chamber has an increased concentration of reactive compounds or substances; and

controlling at least one voltage source of the discharge chamber of the plasma system, the mixing chamber and the conveying means by means of a control and measuring unit for process control, and the control and measuring unit for process control collecting data at least from the discharge chamber of the plasma system, the mixing chamber and the conveying means, which data are used to control the process management.

In an embodiment, a given mixture of fresh compounds or substances is supplied to the mixing chamber from at least one dosing unit via a first supply pipe and the recirculation mass flow is supplied to the mixing chamber via the second supply pipe from the conveying means.

In an embodiment, an output mass flow from the discharge chamber of the plasma system is fed to a recirculation turnout, the recirculation turnout dividing the output mass flow into an effective mass flow and the recirculation mass flow. In an embodiment, the recirculation turnout is monitored by means of the control and measuring unit and regulated depending on the process management.

By means of the control and measuring unit, the temperature in the mixing chamber, the plasma system or the discharge chamber and the chamber may be monitored and regulated as a function of the process control. A voltage source of the plasma system or the discharge chamber may be monitored and regulated by means of the control and measuring unit. The mixing chamber and the chamber may also be monitored and regulated by means of the control and measuring unit.

The assembly according to the invention can be used for decontamination or sterilization of objects, for cleaning objects, and for reduction of harmful compounds or substances, such as organic vapors.

Objects, surfaces or even organic gaseous species are exposed to a very aggressive and oxidizing regime as soon as they are brought into the outlet flow or the recirculation flow of the reactor. With solid objects, depending on the process control and the temperature of the object, condensation may occur on the object surface. In this condensate film, there are high concentrations of H₂O₂ and a low pH value. Such conditions are known to kill germs and oxidize organic substances. This is also used in medical technology and food technology for decontamination or sterilization.

According to an embodiment of the invention, air and water vapor are passed in a suitable manner through a discharge chamber (dielectric barrier discharge) of the plasma system. In addition to short-lived products (ions, radicals, highly excited molecules), this creates various stable species such as nitrogen oxides, ozone and hydrogen peroxide. The recirculation (pumping the outlet of the discharge chamber of the plasma system to the inlet of the discharge chamber of the plasma system) increases the ozone concentration and the concentration of hydrogen peroxide. The pH value in the condensate decreases into the acidic range. With a given initial amount of water and air, recirculation achieves high concentrations of strongly oxidizing species and a degree of acidity that cannot be achieved with a single passage through the discharge chamber of the plasma system. This closed circuit can be opened by adding more air and water and by releasing part of the mass flow at the outlet of the plasma system or the chamber and thus operated continuously.

### Definition

Decontamination, in the context with the present invention, is the removal of dangerous or harmful impurities (contaminations) from objects such as food, clothing, floors, solids, liquids.

The harmful impurities may be of a chemical or biological nature. The term harmful impurities may be understood to comprise germs, spores, fungi, microorganisms of all kinds (viruses, bacteria, fungi, spores, primitive parasites) as well as organic pollutants, toxic organic compounds or poisons. It will be understood by those skilled in the art that the above list is not exhaustive.

### BRIEF DESCRIPTION OF THE DRAWINGS

In the following, exemplary embodiments are intended to explain the invention and its advantages in more detail with reference to the accompanying figures. The size relationships in the figures do not always correspond to the real size relationships, since some shapes are simplified and other shapes are shown enlarged in relation to other elements for better illustration. Reference is made to the accompanying drawings in which:

FIG. 1 shows a schematic view of an embodiment of the invention, wherein in the simplest case the discharge burns within the chamber with the object to be treated;

FIG. 2 shows a schematic view of a further embodiment of the invention, the plasma system being designed as a flow-through reactor;

FIG. 3 shows a schematic view of yet another embodiment of the invention, the plasma system being designed as a flow-through reactor and the chamber being spatially separated from the plasma system; and

FIG. 4 shows a schematic view of a flow chart for carrying out a method for decontaminating or sterilizing at least one object.

### DETAILED DESCRIPTION OF EMBODIMENTS OF THE INVENTION

Identical reference numerals are used for identical or identically acting elements of the invention. Furthermore, for the sake of clarity, only reference numerals are shown in the individual figures which are necessary for the description of the respective figures.

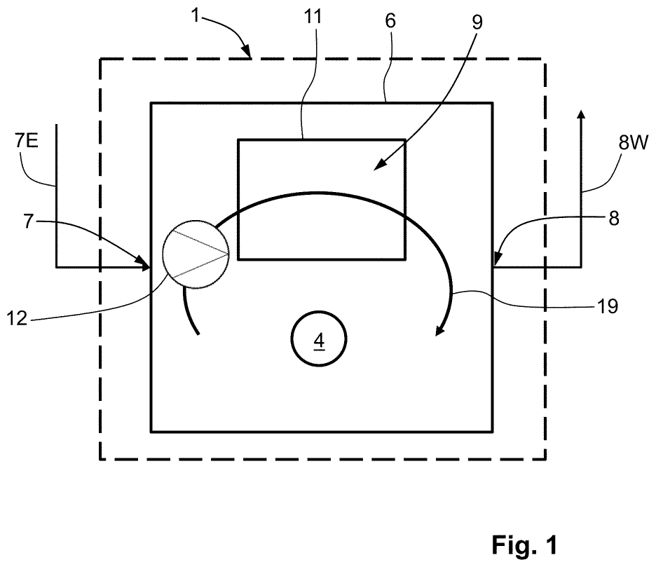

FIG. 1 shows an assembly 1 for decontaminating or sterilizing at least one object 4. In the simplest case, assembly 1, as indicated here, may consist of a chamber 6 in which a discharge 9 is burning. The discharge 9 burns in a plasma system 11 which is arranged inside chamber 6. The at least one object 4 to be sterilized is likewise located in chamber 6. An inlet 7 is associated with chamber 6, via which inlet 7 an inlet mass flow 7E can be brought into chamber 6. Furthermore, an outlet 8 is associated with chamber 6, by means of which outlet 8 an effective mass flow 8W can be transported away from chamber 6. A circulation mass flow 19 is formed within chamber 6, which flows over the plasma system 11 and the object 4 to be sterilized. The circulation mass flow 19 can be set in chamber 6 by means of a conveying means 12. The conveying means 12 may be a pump or a circulating air fan. The circulation mass flow 19 is always guided over the plasma system 11 by conveying means 12 in order to increase the proportions of reactive compounds or substances for the decontamination or sterilization of object 4. Likewise, according to a further embodiment, the circulation mass flow 19 can be maintained via convection. For this purpose, the interior of chamber 6 is designed in such a way that uniform convection is formed. By means of the convection or the conveying means 12 it is ensured that a circulation mass flow 19 with reactive compounds or substances not only reaches the object 4 to be decontaminated or sterilized, but also increases the proportion of reactive compounds or substances per unit volume. According to the structural conditions, the plasma system 11 has a certain length along which the circulation mass flow 19 can be provided with reactive substances. The concentration that can be achieved in a single cycle through the plasma system 11 is not sufficient to carry out an effective decontamination of the objects 4. The concentration of reactive compounds or substances can be increased by means of multiple cycles through the plasma system 11.

Within a certain time, an equilibrium of the concentrations of the reactive compounds or substances is established if the boundary conditions are fixed. If a liquid or aqueous phase and a gaseous phase (air) coexist in chamber 6, the pH value in the aqueous phase falls into the acidic range and the concentration of hydrogen peroxide increases up to a given equilibrium value. By supplying air and water in a suitable ratio via inlet 7, chamber 6 (closed reactor) becomes a flow-through reactor.

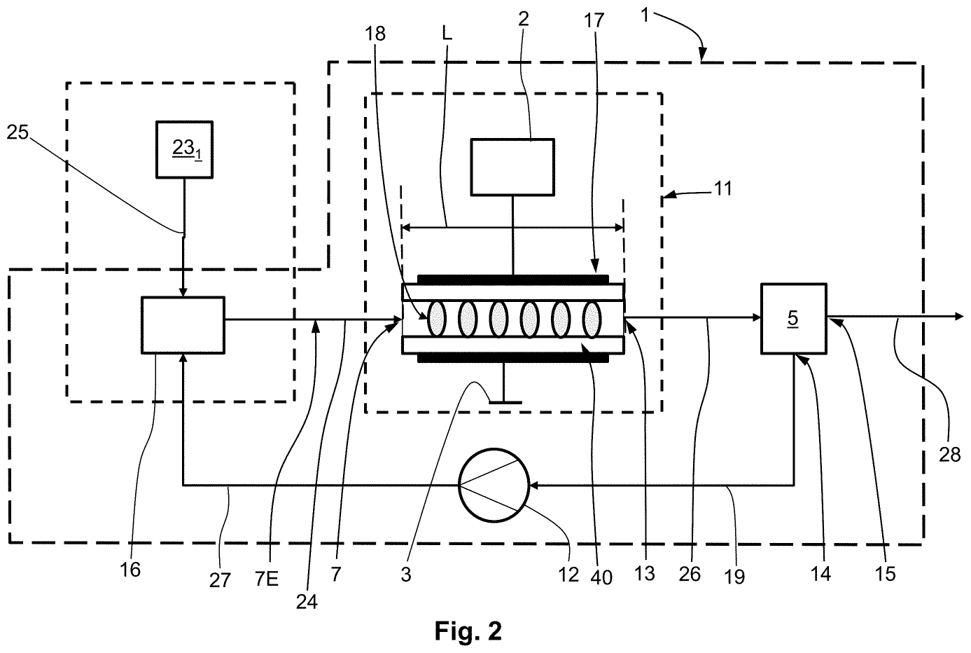

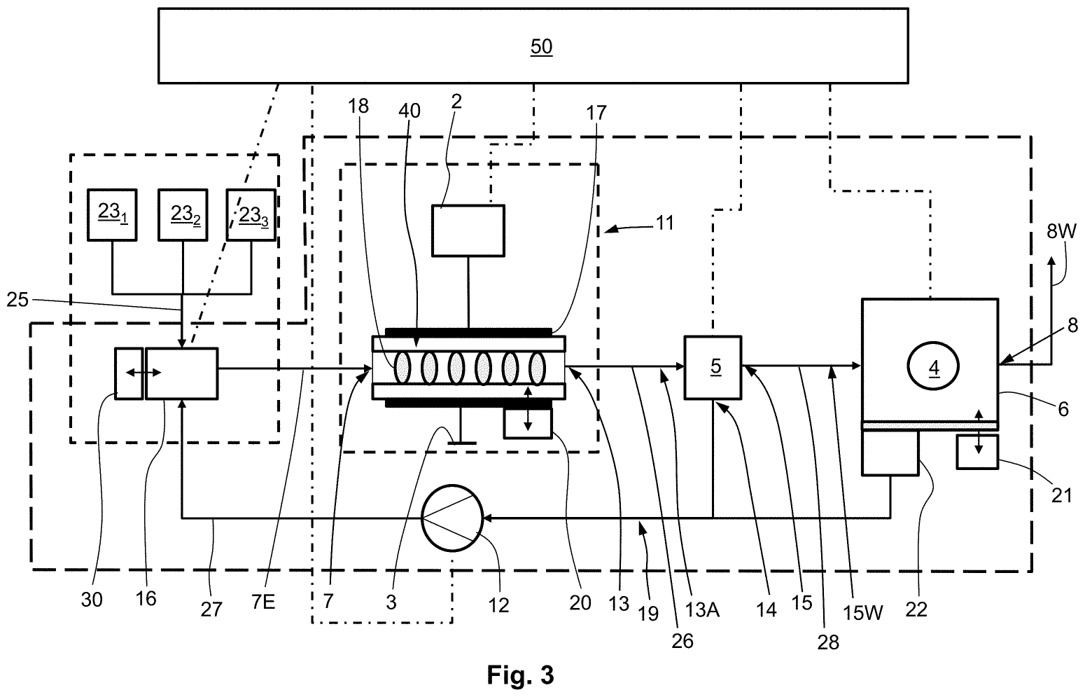

FIG. 2 shows a schematic view of a further embodiment of the invention, the plasma system 11 being designed as a flow-through reactor and the plasma system 11 being spatially separated from chamber 6 (not shown here). At least one mixture of different initial fluid and gaseous components can be fed to plasma system 11 of the assembly 1. In the illustration described in FIG. 2, a single dosing unit 23₁ is provided, which is connected to a mixing chamber 16 via a first supply pipe 25. Furthermore, the mixing chamber 16 is connected to the conveying means 12 via a second supply pipe 27. Although only one single dosing unit 23₁ is shown in the embodiment shown here, this should not be interpreted as a restriction of the invention. As can be seen from the embodiment shown in FIG. 3, more than one dosing unit 23₁, 23₂, . . . 23_{N} may be associated with mixing chamber 16.

The plasma system 11 consists of a voltage source 2 which is connected to a discharge chamber 17. The discharge chamber 17 is connected to a ground connection 3. A discharge zone 18, within which the reactive species are formed, is formed in the discharge chamber 17. The length L of the discharge chamber 17 is decisive for the formation of the proportion or the concentration of the reactive compounds or substances of the mixture leaving the discharge chamber 17.

From the mixing chamber 16, a gas or substance mixture can be fed via a pipe 24 to an inlet 7 of the plasma system 11 or the discharge chamber 17. A gas discharge ignites in the discharge zone 18 of discharge chamber 17. Depending on the intensity of the gas discharge, the gas composition and other process parameters such as flow rate, temperature or pressure, a constant composition of products (reactive compounds or substances) with different lifespans and reactivity that act on the object 4 is created.

The discharge chamber 17 of plasma system 11 is followed by a recirculation turnout 5. The recirculation turnout 5 is fluidically connected to an outlet 13 of the discharge chamber 17 of plasma system 11. A first outlet 14 of recirculation turnout 5 is fluidically connected to the circulation mass flow 19. A second outlet 15 of recirculation turnout 5 is fluidically connected via a pipe 28 to chamber 6 (not shown here). By returning the circulation mass flow 19 into discharge chamber 17 of plasma system 11, the concentration of the reactive compounds or substances can be increased step by step.

FIG. 3 shows a schematic view of yet another embodiment of the invention. Discharge chamber 17 of plasma system 11 is designed as a flow-through reactor. Chamber 6 for the treatment of objects 4 is spatially separated from discharge chamber 17 of plasma system 11. From chamber 6 with object 4, an effective mass flow 8W is emitted to the environment via outlet 8, and reactive compounds or substances flow through chamber 6.

In the embodiment shown here, three dosing units 23₁, 23₂ and 23₃ are connected to mixing chamber 16. The temperature in mixing chamber 16 can be set via a heat sink and/or heat source 30. The input mass flow 7E is fed from mixing chamber 16 to inlet 7 of discharge chamber 17 of plasma system 11. Likewise, the plasma process (generating plasma in discharge chamber 17) itself may be a heat source 30 that is used to vaporize a liquid component, which is supplied by at least one of the dosing units 23₁, 23₂, and 23₃, in mixing chamber 16. The evaporation process in mixing chamber 16 would then be a heat sink 30. This relationship has the advantage that a liquid medium (for example, water or condensate from the recirculation process) can be evaporated without additional heating power.

Typically, the discharge in discharge chamber 17 of plasma system 11 is generated by an electrical excitation that is fed via the voltage source 2 of plasma system 11. For process control, the discharge chamber 17 can be heated or cooled by means of a heat source and/or heat sink 20. In particular, the plasma process in discharge chamber 17 may itself be a heat source 20 which is used to vaporize a liquid component. The evaporation process would then be a heat sink 20. This relationship has the advantage that discharge chamber 17 is cooled by evaporation and a liquid medium (for example, water) can be evaporated without additional heating power. The outlet 13 of discharge chamber 17 of plasma system 11 is fed to the recirculation turnout 5 via a pipe 26. A recirculation mass flow 19 can be fed back from chamber 6 and recirculation turnout 5 to mixing chamber 16 via a second pipe 27 and the conveying means 12.

The recirculation mass flow 19 and thus the recirculation ratio depend on the power of conveying means 12, the setting of recirculation turnout 5 and the settings on dosing units 23₁, 23₂ and 23₃. Second outlet 15 of recirculation turnout 5 is fluidically connected via a pipe 28 to chamber 6 in which the object 4 to be treated is located. Chamber 6 can optionally be kept at a constant temperature via a heat source and/or heat sink 21. If necessary, part of the active gas and the condensate can be introduced into the circulation mass flow 19 from chamber 6 via a condensate separator 22. The chamber 6 flows through the outlet 8 to the environment.

A control and measurement unit 50 is provided for process control, which may be communicatively (wired and/or wireless) connected with the elements of assembly 1, such as voltage source 2, recirculation turnout 5, chamber 6, plasma system 11, conveying means 12, mixing chamber 16, heat sources and/or heat sinks 20, 21, or the at least one dosing unit 23₁, 23₂, . . . , 23_{N}. It is obvious to those skilled in the art that the above list is not exhaustive. Elements of assembly 1 can be switched on or off as required.

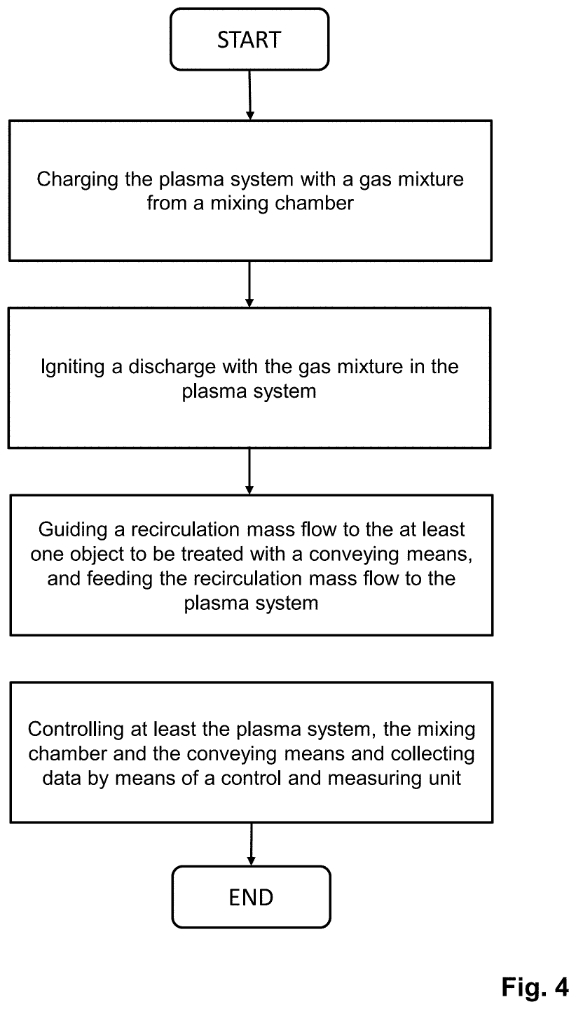

A flow chart of an embodiment of the method according to the invention is shown in FIG. 4. According to the invention, a plasma system 11 or a discharge chamber 17 (flow-through reactor) is charged with at least one gas mixture or mixture from the at least one mixing chamber 16. A discharge is ignited in the discharge chamber 17 of plasma system 11. Depending on the intensity of the gas discharge, the gas composition and other process parameters, such as throughput rate (flow-through rate, flow rate), temperature or pressure results in a constant composition of compounds or substances (products) with different lifetimes and reactivity.

A given mixture of different initial fluid and gaseous components from different dosing units 23₁, 23₂, . . . , 23_{N} is prepared with a mixing chamber 16. The temperature in the mixing chamber 16 can be set via a heat sink and/or heat source 30. The input mass flow 7E is fed into discharge chamber 17 of plasma system 11. In the discharge chamber 17 of plasma system 11, the discharges burn in the discharge zone 18. The discharge is typically generated by an electrical excitation that is fed via the voltage source 2. The plasma system 11 or the discharge chamber 17 can be heated or cooled for process control. The electrical discharge is preferably ignited in the discharge chamber 17 by means of at least one piezoelectric transformer 40. The functioning of a piezoelectric transformer is well known and does not need to be explained again here.

The output mass flow 13A is returned to mixing chamber 16 via recirculation turnout 5 and partly via a conveying means 12. The recirculation mass flow 19 and thus the recirculation ratio depends on the power of the conveying means 12, the setting of the recirculation turnout 5 and the settings of the dosing units 23₁, 23₂, . . . , 23_{N}. The acting mass flow 15W enters a chamber 6 from the second outlet 15 of recirculation turnout 5, in which chamber 6 the object 4 to be treated is located. The chamber 6 can optionally be kept at a constant temperature via a heat source and/or heat sink 22. If necessary, part of the active gas and the condensate can be introduced into the recirculation mass flow 19 via a condensate separator 22. An effective mass flow 8W can be emitted to the environment via an outlet 8.

The control and measuring unit 50 and the principle of recirculation can significantly increase the yield of the reactive species generated in the discharge chamber 17 of plasma system 11. The composition of the mixture can be adjusted over a wide range by means of mixing chamber 16. When a dielectric barrier discharge (for example, piezoelectric transformer 40) is used as the discharge type, the power of the dielectric barrier discharge device can be kept low.

The invention has been described in terms of preferred embodiments. It will be understood by those skilled in the art that changes and modifications of the invention may be made without departing from the scope of the following claims.

### LIST OF REFERENCE NUMBERS

1 Assembly
2 Voltage source
3 Ground connection
4 Object
5 Recirculation turnout
6 Chamber
7 Inlet
7E Input mass flow
8 Outlet
8W Effective mass flow
9 Discharge
11 Plasma system
12 Conveying means, pumping device
13 Outlet
13A Output mass flow
14 First outlet
15 Second outlet
15W Acting mass flow
16 Mixing chamber
17 Discharge chamber
18 Discharge zone
19 Circulation mass flow, recirculation mass flow
20 Heat source, heat sink
21 Heat source, heat sink
22 Condensate separator
23₁, 23₂, . . . , 23_{N} Dosing unit
24 Pipe
25 First supply pipe
26 Pipe
27 Second supply pipe
28 Pipe
30 Heat sink and/or heat source
40 Piezoelectric transformer
50 Control and measuring unit
L Length

What is claimed is:

1. An assembly for decontaminating or sterilizing at least one object, comprising a chamber in which the at least one object is placed and a plasma system which is fluidically connected with the chamber, the assembly comprising:
a mixing chamber, which is arranged upstream of the plasma system and connected via a pipe to an inlet of a discharge chamber of the plasma system, such that an input mass flow can be supplied to the discharge chamber of the plasma system;
a conveying means which is arranged in a second supply pipe from the chamber to the mixing chamber in order to guide a circulation mass flow from the chamber back to the mixing chamber; and
a recirculation turnout provided between the plasma system and the chamber, wherein the recirculation turnout is fluidically connected with an outlet of the plasma system via a pipe, a first outlet of the recirculation turnout is fluidically connected with the circulation mass flow, and a second outlet of the recirculation turnout is fluidically connected with the chamber via a pipe.

2. The assembly according to claim 1, wherein a heat source and/or heat sink is associated with the plasma system and/or a heat source or heat sink is associated with the chamber.

3. The assembly according to claim 1, wherein a condensate separator is associated with the chamber and the second supply pipe leads from the condensate separator to the mixing chamber.

4. The assembly according to claim 1, wherein the mixing chamber is connected with the conveying means via a first supply pipe, and the mixing chamber is connected with at least one dosing unit via the second supply pipe.

5. The assembly according to claim 4, wherein a heat source and/or heat sink is assigned to the mixing chamber.

6. The assembly according to claim 1, wherein a control and measuring unit is provided, which is communicatively connected at least with a voltage source of the plasma system, the recirculation turnout, the chamber, the conveying means, the mixing chamber, the heat sources and/or heat sinks or the at least one dosing unit.

7. The assembly according to claim 1, wherein the plasma system comprises a discharge chamber, in which at least one piezoelectric transformer is provided which is connected to the voltage source of the plasma system for generating reactive species.

8. A method for decontaminating or sterilizing objects, comprising the following steps:
charging a discharge chamber of a plasma system with a gas mixture from at least one mixing chamber via a pipe;
igniting a discharge with the gas mixture in the discharge chamber of the plasma system;
feeding an acting mass flow from the discharge chamber to a chamber via pipes;
guiding a recirculation mass flow from the chamber to the mixing chamber with a conveying means in a second supply pipe, and feeding again the recirculation mass flow to the chamber via the discharge chamber of the plasma system, so that an acting mass flow leaving the discharge chamber has an increased concentration of reactive compounds or substances;
feeding an output mass flow from the discharge chamber of the plasma system to a recirculation turnout, wherein the recirculation turnout divides the output mass flow into the acting mass flow and the recirculation mass flow;
controlling at least one voltage source of the discharge chamber of the plasma system, the mixing chamber and the conveying means by means of a control and measuring unit for process control, and the control and measuring unit for process control collecting data at least from the discharge chamber of the plasma system, the mixing chamber and the conveying means, which data are used to control the process management, wherein the control and measuring unit also monitors and regulates the recirculation turnout.

9. The method according to claim 8, wherein the mixing chamber receives a predetermined mixture from at least one dosing unit and the recirculation mass flow from the conveying means via a second supply pipe.

10. The method according to claim 8, wherein the temperature in the mixing chamber, in the discharge chamber of the plasma system and the chamber is monitored and regulated by means of the control and measuring unit.

11. The method according to claim 8, wherein the discharge is ignited with the gas mixture supplied from the mixing chamber by means of at least one piezoelectric transformer in the discharge chamber of the plasma system.
